(19) United States
(12) Patent Application Publication
(10) Pub. No.: US 2010/0273166 A1
(43) Pub. Date: Oct. 28, 2010

(54) BIOSENSOR DEVICE AND METHOD OF SEQUENCING BIOLOGICAL PARTICLES

(75) Inventor: Pablo Garcia Tello, Leuven (BE)

Correspondence Address: NXP, B.V.;NXP INTELLECTUAL PROPERTY & LICENSING, M/S41-SJ, 1109 MCKAY DRIVE, SAN JOSE, CA, 95131, US

(73) Assignee: NXP B.V., Eindhoven (NL)

(21) Appl. No.: 12/747,796

(22) PCT Filed: Dec. 4, 2008
(86) PCT No.: PCT/IB08/55088

§ 371 Date: Jun. 11, 2010

(30) Foreign Application Priority Data
Dec. 13, 2007 (EP) 07123156.7

(51) Int. Cl. C12Q 1/68; C12M 1/34

(52) U.S. Cl. 435/6; 435/287.2

(57) ABSTRACT

A biosensor device (100) for sequencing biological particles (102), the biosensor device (100) comprising at least one substrate (104), a plurality of sensor active regions (106) provided on each of the at least one substrate (104) and each comprising a primer (108) having a sequence being complementary to a part of a sequence of the biological particles (102) and enabling generation of fragments having a sequence being inverse to a part of the sequence of the biological particles (102) at the primer (108), and a determination unit (114) adapted for individually determining a size of the fragments generated at the primer (108) of each of the plurality of sensor active regions (106), the fragment replication being terminated in the presence of replication terminating sequence units (116 to 119).

### FIELD OF THE INVENTION

The invention relates to a biosensor device.

Moreover, the invention relates to a method of sequencing biological particles.

### BACKGROUND OF THE INVENTION

A biosensor may be denoted as a device to be used for the detection of an analyte that combines a biological component with a physicochemical or physical detector component.

For instance, a biosensor may be based on the phenomenon that capture molecules immobilized on a surface of a biosensor may selectively hybridize with target molecules in a fluidic sample, for instance when an antibody-binding fragment of an antibody or the sequence of a DNA single strand as a capture molecule fits to a corresponding sequence or structure of a target molecule. When such hybridization or sensor events occur at the sensor surface, this may change the electrical properties of the surface, which can be detected as the sensor event.

DNA sequencing is an important application in biochemistry. The term DNA sequencing encompasses biochemical methods for determining the order of the nucleotide bases, adenine, guanine, cytosine, and thymine, in a DNA oligonucleotide. The sequence of DNA constitutes the heritable genetic information in nuclei, plasmids, mitochondria, and chloroplasts that forms the basis for the developmental programs of all living organisms. Determining the DNA sequence is therefore useful in basic research studying fundamental biological processes, as well as in applied fields such as diagnostic or forensic research.

The Sanger method is a conventional method for DNA sequencing and is an enzymatic method for determining the nucleotide sequence of a fragment of DNA. However, the Sanger method conventionally relies on the use of labels to perform DNA sequencing. It further suffers from the limitations of the gel electrophoresis method being performed in the context of the conventional Sanger method.

### OBJECT AND SUMMARY OF THE INVENTION

It is an object of the invention to provide a simple system for sequencing biological particles.

In order to achieve the object defined above, a biosensor device and a method of sequencing biological particles according to the independent claims are provided.

According to an exemplary embodiment of the invention, a biosensor device for sequencing (that is for determining a sequence of) biological particles is provided, the biosensor device comprising at least one substrate (for instance one substrate, four substrate, or any other number of substrates), a plurality of sensor active regions provided on each of the at least one substrate and each comprising a primer having a sequence being complementary (or inverse) to a part of a sequence of the biological particles and enabling generation of fragments having a sequence being inverse (or complementary) to a part of the sequence of the biological particles at the primer (particularly in the presence of sequence units of the sequence of the biological particles and in the presence of a replication enzyme), and a determination unit (for instance a processor) adapted for individually determining a size (which may be indicative of a length, a mass, a mass distribution, a moment of inertia, etc.) of the fragments replicated (or generated) at the primer of each of the plurality of sensor active regions, the fragment generation being terminated (or finished) in the presence of replication terminating sequence units.

According to another exemplary embodiment of the invention, a method of sequencing biological particles is provided, the method comprising providing a plurality of sensor active regions on each of at least one substrate, each of the plurality of sensor active regions comprising a primer having a sequence being complementary to a part of a sequence of the biological particles and enabling generation of fragments having a sequence being inverse to a part of the sequence of the biological particles at the primer (particularly in the presence of sequence units of the sequence of the biological particles and in the presence of a replication enzyme), and individually determining a size of fragments replicated at the primer of each of the plurality of sensor active regions, the fragment generation being terminated in the presence of replication terminating sequence units.

The term “biosensor” may particularly denote any device that may be used for the detection of a component of an analyte comprising biological molecules such as DNA, RNA, proteins, enzymes, cells, bacteria, virus, etc. A biosensor may combine a biological component (for instance capture molecules at a sensor active surface capable of detecting molecules) with a physicochemical or physical detector component (for instance a capacitor having a capacitance which is modifiable by a sensor event, or a beam being mechanically modifiable by a sensor event). Such a biosensor may fulfil the task of determining a sequence, that is to say an order of constituents, of a biological particle.

The term “sequencing” may particularly denote determining the sequence of biological particles, which is a succession of basic units from which the biological particles are constituted. Examples for such basic units are nucleobases (or nucleotide bases) of a DNA sequence or amino acids of a protein. The sequence of a DNA or RNA molecule is the linear order of nucleotides along the DNA or RNA molecule, and the process of obtaining this may be denoted as sequencing. Genome sequencing may aim to generate the linear order of all nucleotides present in the nuclear DNA of an organism.

The term “biosensor chip” may particularly denote that a biosensor is formed as an integrated circuit, that is to say as an electronic chip, particularly in semiconductor technology, more particularly in silicon semiconductor technology, still more particularly in CMOS technology. A monolithically integrated biosensor chip has the property of very small dimensions thanks to the use of micro-processing technology, and may therefore have a large spatial resolution and a high signal-to-noise ratio particularly when the dimensions of the biosensor chip or more precisely of components thereof approach or reach the order of magnitude of the dimensions of biomolecules.

The term “sensor active region” may particularly denote an exposed region of a sensor, which may be brought in interaction with a fluidic sample so that a detection event may occur in the sensor active region. In other words, the sensor active region may be the actual sensitive area of a sensor device, in which area processes take place that form the basis of the sensing. A corresponding sensing principle may be an electrical sensing principle (that is a change of the electric properties of the sensor active region), a mechanical sensing principle (that is a change of the mechanical properties of the sensor active region), or an optical sensing principle (that is a change of the optical properties of the sensor active region).

The term “substrate” may denote any suitable material, such as a semiconductor, glass, plastic, etc. According to an exemplary embodiment, the term “substrate” may be used to define generally the elements for layers that underlie and/or overlie a layer or portions of interest. Also, the substrate may be any other base on which a layer is formed, for example a semiconductor wafer such as a silicon wafer or silicon chip. Several different substrates may be separate bodies with or without a mechanical connection between different substrates.

The term “fluidic sample” may particularly denote any subset of the phases of matter. Such fluids may include liquids, gases, plasmas and, to some extent, solids, as well as mixtures thereof. Examples for fluidic samples are DNA containing fluids, blood, interstitial fluid in subcutaneous tissue, muscle or brain tissue, urine or other body fluids. For instance, the fluidic sample may be a biological substance. Such a substance may comprise proteins, polypeptides, nucleic acids, DNA strands, etc.

The term “biological particles” may particularly denote any particles that play a significant role in biology or in biological or biochemical procedures, such as genes, DNA, RNA, proteins, enzymes, cells, bacteria, virus, etc.

The term “primer” may particularly denote a short sequence of basic units from which a replication of biological particles can initiate. Such a short sequence may be a sequence of amino acids or of nucleobases. From a short sequence of nucleobases, DNA replication can initiate.

The term “complementary sequence” or “inverse sequence” may particularly denote that a corresponding sequence of basic units of the primer and a sequence of the biological particles that are inverse to one another. For instance, adenine is inverse or complementary to thymine, and guanine is inverse or complementary to cytosine.

The term “sequence units” may particularly denote basic building blocks or constituents of biological particles, of a primer or of fragments, in the case of DNA replication the nucleobases adenine (A), guanine (G), cytosine (C), thymine (T).

The term “replication enzyme” may particularly denote an enzyme in the presence of which a replication of a nucleobase sequence can be promoted. An example for such a replication enzyme of DNA is the DNA polymerase.

The term “fragment” may particularly denote a sequence of basic building blocks formed starting from the primer and aligned to a portion of the biological particle that is complementary to the primer.

The term “replication terminating sequence unit” may particularly denote molecules (such as dideoxynucleotides) being chemically slightly modified as compared to the basic building blocks. However, when a replication terminating sequence unit is present in an environment of the current fragment end growing at the biological particle and when such a replication terminating sequence unit is complementary to an exposed portion of the biological particle at the current fragment end, such a replication terminating sequence unit may be added to the end of the fragment and terminates the fragment formation. Hence, after the alignment of this replication terminating sequence unit at the biological particle to be sequenced, the replication procedure is terminated and no further basic building block may be added to the replication terminating sequence unit. In the example of DNA replication, ddT, ddC, ddG and ddA may be denoted as replication terminating sequencing units regarding thymine (T), cytosine (C), guanine (G), and adenine (A), respectively.

According to an exemplary embodiment of the invention, a modified label free miniaturized Sanger sequencing system may be provided for performing sequencing with a substrate bound biosensor device. In such an embodiment, a plurality of sensor active regions may be supplied with immobilized primers and biological particles under analysis so that upon the addition of sequence units, replication enzyme and replication terminating sequence units (which shall be different for different portions of the substrate or for different substrates) replication starts and is terminated at specific portions in accordance with the sequence of the biological particles and in accordance with the specific replication terminating sequence units. In other words, for each of the portions of the substrate or for each substrate, an assigned replication terminating sequence unit may be present, so that fragments of specific lengths may be generated characteristically in each portion, wherein the fragments have at an end portion the replication terminating sequence unit. When different replication terminating sequence units are used for different portions of the substrate, the set of fragments present in the different portions of the substrate may allow to derive information regarding the sequence of the biological particles. The combination of the fragment set information (particularly the combination of fragment lengths in a set with the corresponding replication terminating sequence unit) from the different portions of the substrate may then serve to derive or reconstruct the entire sequence of the biological particles. According to an exemplary embodiment of the invention, the presence of these fragments and their length or other quantity characteristics may be sensed at the different sensor active regions, wherein in dependence of the length an electrical, mechanical or other physical parameter at this specific sensor active region is characteristically modulated, thereby allowing to measure with a sensor array all present fragments to reconstruct the sequence of the biological particles.

More specifically, a method for DNA sequencing is provided which allows to perform label-free DNA sequencing using a technology that can be fabricated using conventional CMOS processing. Embodiments of the invention apply a modified Sanger method wherein DNA synthesis is done by an enzyme (DNA polymerase) that adds nucleotides (A, T, G, C) to the 3′-end of a primer DNA chain towards the 5′ end. It is possible to stop the polymerase (that is DNA replication) reaction when using dideoxynucleotides. Dideoxynucleotides are almost identical to the normal nucleotides. Addition of a dideoxynucleotide to the 3′-OH end of a DNA chain stops the action of the polymerase and terminates chain elongation. The dideoxy sequencing (also called chain termination or Sanger method) uses an enzymatic procedure to synthesize DNA chains of varying length, stops DNA replication at one of the four bases and then determines the resulting fragment length.

Each sequencing reaction substrate (a ddT substrate, a ddC substrate, a ddG substrate, and a ddA substrate) may contain:
a collection of DNA templates (unknown sequence), a collection of primer sequences (one per template), and a DNA polymerase (one per primer and template) to initiate synthesis of a new strand of DNA at the point where the primer is hybridized to the template;
a sufficiently high concentration of the four nucleotides (A, T, C and G) to extend the DNA primer strand complementary to the template;
a low concentration of (exactly) one of the four dideoxynucleotides, which terminates the growing chain wherever it is incorporated. For instance, substrate portion ddA has ddA, substrate portion ddC has ddC, substrate portion ddG has ddG, substrate portion ddT has ddT.

As an example, it will be explained what is happening at the substrate portion named ddT. The polymerase starts adding nucleotides along the primer that are complementary to the DNA template until it incorporates a ddT. Then it stops. The result in the ddT portion of the substrate is a collection of fragments of different lengths of the DNA template ending always with a ddT. The result of the other three substrate portions will be analogous except that all fragments ends in ddA, ddG or ddC, respectively.

By arranging sensor active regions on and/or in the substrate(s) and by reading out the information from the substrate(s) by the determination unit, embodiments of the invention allow for DNA sequencing which is label-free and which does not suffer from limitations of an electrophoresis method (required for conventional Sanger method), and also allows to increase the level of parallelism.

Embodiments of the invention therefore provide a biosensor device which is miniaturized and which has substrate bound sensor active regions which electrically, optically or mechanically allow to detect signals which are indicative of the length/mass/dimension of a specific fragment terminated at a specific portions of the biological particles depending on the replication terminating sequence units which are present at different substrate portions, thereby allowing to have information regarding different basic units from the different substrate portions.

When carrying out such a modified Sanger method in accordance with an embodiment of the invention, the concentration ratio of dideoxynucleotide to a normal nucleotide may be 1:100. With this, it may be assured that the incorporation of ddT for example is a “rare but not too rare” event, so indeed chains of different lengths may be obtained. Furthermore, it may be ensured by such a concentration adjustment that there is a time when the polymerase reaction stops because all or most of the ddTs are consumed.

With a sufficient number of runs (or with a large number of electrodes in a single run), it may be ensured that it is possible to receive all combinations of nucleotides incorporated to a primer so that it is possible to reconstruct the sequence accurately. Furthermore, it is possible to get repeated combinations at the electrodes so as to produce enough redundant measurements to avoid any errors in reading.

For example, it is possible to take into account that for example on a first chip or substrate only the combinations ending in a ddA can occur (on a second chip or substrate the ones ending in a ddT, on a third chip or substrate only the ones ending in a ddG, on a fourth chip or substrate only the ones ending in a ddC) and that these combinations are dictated by the unknown nucleotide sequence in the template. The primer just reconstructs base per base the complementary sequence of the template.

Next, further exemplary embodiments of the biosensor device will be explained. However, these embodiments also apply to the method.

The determination unit may be adapted for determining the sequence of the biological particles based on the individually determined size of the fragments considering information regarding an assigned type of replication terminating sequence units. In other words, different substrates or different substrate portions may be assigned to unique replication terminating sequence units, for instance to an assigned one of the four dideoxynucleotides (ddA, ddT, ddG, ddC). Then, one can be sure that at each substrate or substrate portion, the generated fragments end with the only one of the four dideoxynucleotides added to this specific substrate or substrate portion. This allows deriving, from each substrate or substrate portion, unambiguous information regarding to a specific one of the four nucleobases in the sequence of the biological molecule. Each sensor active region may then determine the dimension or length or mass or number of nucleotides at the specific portion based on the detection of an electrical, optical or mechanical signal. The combination of the different substrates or substrate portions may then allow to unambiguously determining the sequence of the biological particles, for instance a DNA sequence.

The at least substrate may consist of exactly one substrate having delimited compartments, particularly four delimited compartments, each of the delimited compartments comprising a plurality of the sensor active regions and being assigned to a unique type of the replication terminating sequence units. When four compartments, that is spatially delimited regions, are provided, different cavities may be formed in which the corresponding experiment with a specific replication terminating sequence unit may be carried out. With spatially delimited compartments, it may be ensured that no mixing between different fluidic samples including different replication terminating sequence units occurs. This may avoid undesired crosstalk.

Alternatively, the at least one substrate may comprise a plurality of separate substrates, particularly four separate substrates, each of the separate substrates comprising a plurality of the sensor active regions and being assigned to a specific type of the replication terminating sequence units. Particularly, four different biosensor chips may be used for different replication terminating sequence units, that is for the four different dideoxynucleotides (ddA, ddC, ddG, ddT).

As a further alternative, it is possible to perform the experiments on a single substrate and to bring the substrate, at a time, only in contact with a specific type of replication terminating sequence units. After such a partial experiment, the biosensor device or the substrate may be rinsed to made ready for a next step or procedure in which another replication terminating sequence unit is tested. Such a procedure, which may be repeated four times, allows to derive information regarding the different basic units/replication terminating sequence units one after the other. In the case of DNA sequencing, four experiments have to be performed one after the other.

In an embodiment, the plurality of sensor active regions may comprise electrodes. With electrodes, electrical signals may be measured in an environment of the electrodes, wherein the replication or generation of the fragments may characteristically modify the electrical properties, for instance the capacitance, in an environment of the electrodes. In such an embodiment, the primers may be immobilized at the electrodes and the biological particles may hybridize with the primers. Then, a fragment generation may be triggered and the set of fragments may be identified by the presence of the replication terminating sequence units at the end of the exposed portion of the biological particles which may characteristically modify the electrical properties of the electrodes.

The determination unit (which may be an integrated circuit) may be provided with the electric signals received from the electrodes, the electrical signals being indicative of the assigned size of fragments since the fragment size may modify the dielectric properties in an environment of the electrodes. Particularly, the determination unit may carry out an algorithm which allows to retrieve or derive a fragment length from the electrical signals. In combination with the knowledge of the corresponding replication terminating sequence units, information regarding a specific basic unit can be derived at a specific position along the DNA sequence. The combination of the electrode signals may then allow to derive the entire sequence of the biological particles.

Alternatively, the plurality of sensor active regions may comprise cantilevers, particularly nanocantilevers, being bendable in a characteristic manner in accordance with the size of fragments. When the primers are immobilized at the cantilevers, generation of fragments coupled to the cantilevers may change the mechanical load acting on the bendable cantilevers by effecting a torsional moment. Therefore, a mechanical bending signal may be electrically sensed, or may be sensed optically due to a modified deflection of a laser beam. Such cantilevers may be MEMS structures (micro-electromechanical structures), thereby increasing the accuracy of the system. It may be appropriate to align the cantilevers horizontally to promote the bending due to the grown fragments under the influence of the gravitational force.

Still referring to the cantilever embodiment, the determination unit may be adapted for sampling a bending of the cantilevers using an electromagnetic radiation beam, particularly a laser beam, wherein a deflection of the electromagnetic radiation beam at the bendable cantilevers may be indicative of the assigned size of fragments. The larger the fragment, the more will the cantilever be bent under the mechanical load of the fragments. Therefore, a changed reflectance or deflectence characteristic of the electromagnetic radiation beam, particularly of a light beam, may be detected and may allow to calculate the size, mass or length of the corresponding fragment. With different replication terminating sequence units in different portions of the biosensor device, different fragments may be sensed in each of these portions. The knowledge of the specific type of a replication terminating sequence unit in a specific portion may then allow to assign a corresponding bending to a corresponding fragment of the biological particles, thereby yielding information regarding the sequence of the biological particles.

The cantilevers may be nanocantilevers. The term “nanocantilevers” may particularly denote the fact that cantilevers may have at least one dimension in the order of magnitude of nanometres to tenth of nanometres or hundreds of nanometres, or less. For instance, such nanocantilevers may be carbon nanotubes.

When the sensor active region comprises a nanoelectrode, the dimensions of the electrode may be in the order of magnitude of nanometers, for instance may be less than 300 nm, for instance may be less or equal than 250 nm, or may be less or equal than 130 nm. The smaller the nanoelectrodes, the more sensitive the resulting sensor region.

The nanoelectrode may comprise copper material, particularly copper material being covered by a self-assembled monolayer (SAM). These materials may serve as oxidation protection layers or as barrier layers or for enabling bonding of capture molecules, thereby allowing to implement the relative sensitive material copper which is highly appropriate due to its high electrical conductivity and compliance with procedural requirements. Copper material has chemically similar properties to gold which is conventionally used in biosensing, but which has significant disadvantages because it diffused rapidly into many materials used in silicon process technology, thereby deteriorating the IC's performance, it is difficult to etch, and gold residues are hard to remove in cleaning steps. However, alternative embodiments of the invention may involve gold as well. Furthermore, materials such as aluminium or the like may be used as well.

The biosensor may comprise an electrically insulating layer forming part of a surface of the biosensor chip and having a recess, wherein an exposed surface of the sensor active region is provided as a sensing pocket in the recess. By providing sensing pockets, shielded and defined regions may be formed in which a sensor event may take place. In the bottom of the recess, a nanoelectrode may be provided with small dimensions, so that a high sensitivity may be achieved. Therefore, the biosensor chip may be used even under harsh conditions.

The biosensor device may be manufactured in MEMS (microelectromechanical structure) technology. MEMS generally range in size from a micrometer (a millionth of a meter) to a millimeter (thousandth of a meter). By such a technique, it is for instance possible to manufacture cantilever beams which are sufficiently sensitive with regard to replicated fragments.

The biosensor chip may be manufactured in CMOS technology. CMOS technology, particularly the latest generations thereof, allow to manufacture structures with very small dimensions so that (spatial) accuracy of the device will be improved by implementing CMOS technology particularly in the Front End of the Line. A CMOS process may be a preferred choice. A BiCMOS process in fact is a CMOS process with some additional processing steps to add bipolar transistors. The same holds for CMOS processes with other embedded options like embedded flask, embedded DRAM, etc. In particular this may be relevant because the presence of an option often provides opportunities to use additional materials that come with the options “at zero cost”. For instance, an appropriate high-k material (an insulating material with a high dielectric constant, for example aluminium-oxide) that comes with an embedded DRAM process can be used “at zero cost” to cover the copper surface of the nanoelectrodes with a protective dielectric layer on which, subsequently, a SAM can be deposited (the function of the SAM would be to “functionalize” that sensor surface, for instance to be able to attach capture probe molecules).

The biosensor may comprise a switch transistor structure formed in the Front End of the Line and electrically coupled to the sensor active region. Such a switch transistor may be a field effect transistor realized as an n-MOSFET or a p-MOSFET. The sensor active surface may be electrically coupled to one of the source/drain regions of such a switch transistor structure, so that a readout voltage applied to the gate of the transistor may result in a source/drain current which depends on the presence or absence (and also on the amount) of the particles of the fluidic sample, since this may have an impact on the voltage of the capacitor which may be transferred to one of the source/drain regions. Alternatively, such a voltage may directly act on the gate region of a MOSFET, thereby changing the threshold voltage or changing the value of a current flowing between source and drain when a voltage is applied in between.

An exposed surface of the sensor active region may have a dimension of at most 1.6 times, particularly of at most 1.1 times, more particularly of at most 0.7 times, of a minimum lithographic feature size of a CMOS process applied for manufacturing the biosensor chip. Particularly, a biosensor may be provided that has a bio-sensitive part made at the surface of a Back End of the Line portion of an advanced CMOS process with copper interconnect, where the diameter of the exposed copper surface is equal to or smaller than 1.6 times the minimum lithographic feature size of the smallest copper via holes of the corresponding CMOS process. A value slightly less than 1 (for instance down to about 0.7) may correspond to sub-feature size holes made by adding minor additional processing steps, or by applying a first-metal feature size. This would require some additional processing steps or more stringent control over more demanding standard CMOS steps (for instance in case of applying first-metal feature size). Even smaller values can be made in principle, but would require extensive additional processing effort. Furthermore, they would lead to a significantly reduced fraction of sensitive area of a biosensor cell. Also, the sensitivity of the sensor would not improve significantly by decreasing the radius even more because the total capacitance of the nanoelectrode sensor node would be limited by parasitic capacitances anyway. To be able to really benefit from smaller nano-electrode radii it would be necessary to decrease the dimensions of the transistors and interconnect layers as well, that is to say stepping to the next CMOS node.

The biosensor device may be monolithically integrated in a semiconductor substrate, particularly comprising one of the group consisting of a group IV semiconductor (such as silicon or germanium), and a group III-group V semiconductor (such as gallium arsenide).

The biosensor chip or microfluidic device may be or may be part of a sensor device, a sensor readout device, a lab-on-chip, a sample transport device, a sample mix device, a sample washing device, a sample purification device, a sample amplification device, a sample extraction device or a hybridization analysis device. Particularly, the biosensor or microfluidic device may be implemented in any kind of life science apparatus.

For any method step, any conventional procedure as known from semiconductor technology may be implemented. Forming layers or components may include deposition techniques like CVD (chemical vapour deposition), PECVD (plasma enhanced chemical vapour deposition), ALD (atomic layer deposition), or sputtering. Removing layers or components may include etching techniques like wet etching, plasma etching, etc., as well as patterning techniques like optical lithography, UV lithography, electron beam lithography, etc.

Embodiments of the invention are not bound to specific materials, so that many different materials may be used. For conductive structures, it may be possible to use metallization structures, silicide structures or polysilicon structures. For semiconductor regions or components, crystalline silicon may be used. For insulating portions, silicon oxide or silicon nitride may be used.

The biosensor may be formed on a purely crystalline silicon wafer or on an SOI wafer (Silicon On Insulator).

Any process technologies like CMOS, BIPOLAR, BICMOS may be implemented.

The aspects defined above and further aspects of the invention are apparent from the examples of embodiment to be described hereinafter and are explained with reference to these examples of embodiment.

### BRIEF DESCRIPTION OF THE DRAWINGS

The invention will be described in more detail hereinafter with reference to examples of embodiment but to which the invention is not limited.

FIG. 1 illustrates a biosensor device for sequencing DNA according to an exemplary embodiment of the invention.

FIG. 2 to FIG. 5 show schemes illustrating a conventional Sanger method.

FIG. 6 shows a plan view of a biosensor device according to an exemplary embodiment of the invention.

FIG. 7 shows a cross-sectional view of a monolithically integrated portion of a sensor device according to an exemplary embodiment of the invention.

FIG. 8 to FIG. 10 show experimental images of a biosensor device manufactured in accordance with embodiments of the invention.

FIG. 11 illustrates an enlarged portion of a sensor active region of a biosensor device according to an exemplary embodiment of the invention.

FIG. 12 illustrates different substrates of a biosensor device according to an exemplary embodiment of the invention.

FIG. 13 illustrates an array of sensor active regions of a biosensor device according to an exemplary embodiment of the invention and the corresponding information derived thereof.

FIG. 14 to FIG. 17 schematically illustrate a way how information regarding a specific nucleotide base of a DNA sequence can be derived from each of the individual substrates shown in FIG. 12.

FIG. 18 schematically illustrates how a DNA sequence may be derived from the information derived from FIG. 14 to FIG. 17.

FIG. 19 illustrates a biosensor device of a cantilever type according to an exemplary embodiment of the invention.

FIG. 20 illustrates how DNA sequence information can be derived from a bending of the cantilevers of FIG. 19.

FIG. 21 shows a sensor device according to an exemplary embodiment of the invention having a plurality of substrates each carrying a plurality of cantilevers.

FIG. 22 schematically illustrates how information can be derived from individual ones of the cantilevers of a structure as shown in FIG. 21.

### DESCRIPTION OF EMBODIMENTS

The illustration in the drawing is schematical. In different drawings, similar or identical elements are provided with the same reference signs.

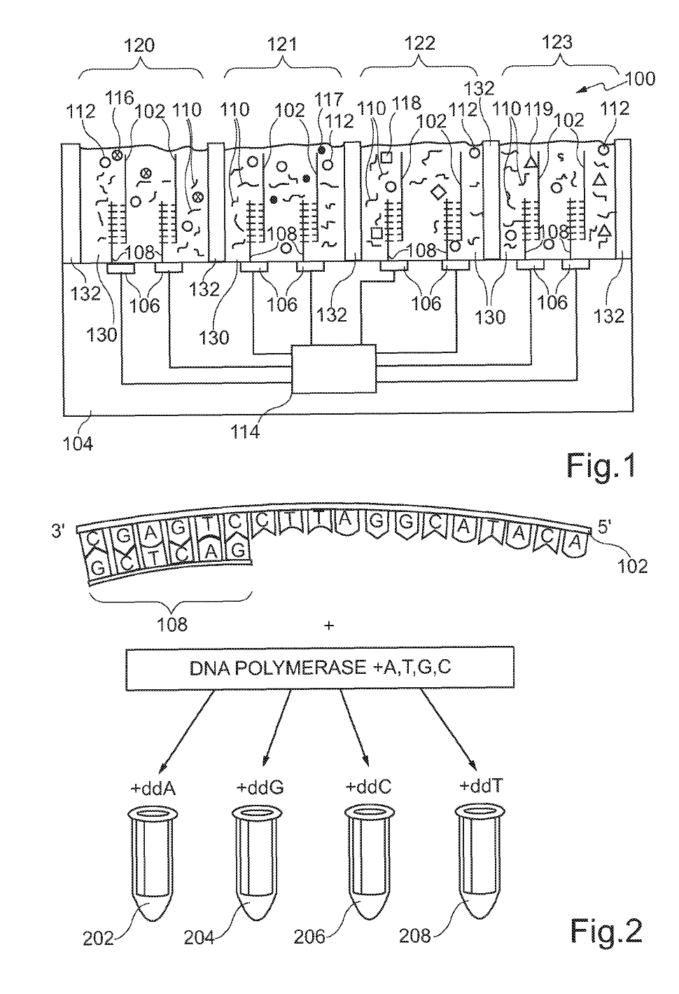

In the following, referring to FIG. 1, a biosensor device 100 for sequencing DNA molecules 102 according to an exemplary embodiment of the invention will be explained.

The biosensor device 100 comprises a silicon substrate 104. A plurality of sensor active regions 106 is provided on a surface of the silicon substrate 104. On each of the sensor active regions 106, a primer molecule 108 is immobilized, which is an oligonucleotide being complementary to an end portion of the DNA sequence 102. The primer 108 has a sequence that is complementary to an end of the sequence of the biological particles 102. Thus, an upper portion of the DNA 102 remains exposed to a fluidic environment 130 in which nucleotide bases 110 (A, T, C, and G) are present as well as a DNA polymerase 112 as a replication enzyme.

Electrical signals detected by each of the plurality of sensor active regions 106, nanoelectrodes in the embodiment of FIG. 1, may be supplied to a central processing unit 114 or any other entity having processing capabilities which may be provided as a monolithically integrated circuit in the silicon substrate 104 (alternatively provided apart from the substrate 104, for instance as a separate electronic circuit). The determining unit 114 is adapted for individually determining a size of fragments (not shown in FIG. 1) replicated at the primer 108 of each of the plurality of sensor regions 106, wherein the fragment replication is terminated at a characteristic portion of the DNA 102 in each of individual compartments 120 to 123, since different dideoxynucleotides 116 to 119 are present in each of the compartments 120 to 123.

More particularly, the determination unit 114 is adapted for determining the sequence of the DNA 102 based on the individually determined sizes of the fragments considering information regarding an assigned type of dideoxynucleotides in each of the compartments 120 to 123.

In the embodiment of FIG. 1, only a single silicon substrate 104 is provided having the delimited compartments 120 to 123 (delimited by separation walls 132). Each of the delimited compartments 120 to 123 comprises a plurality of the sensor active regions 106 and is assigned to a specific type of the dideoxynucleotides. Particularly, the compartment 120 comprises ddA 116, the compartment 121 comprises ddC 117, the compartment 122 comprises ddG 118, and the compartment 123 comprises ddT 119. Thus, in dependence on the sequence of the DNA 102, the fragments formed at the exposed portions of the DNA 102 uncovered by the primer 108 depends on the DNA sequence and the corresponding dideoxynucleotides 116. Since a number of fragments in accordance with the DNA molecule 102 sequence is formed in each of the compartments 120 to 123 and the length of the fragments can be detected based on the electric signals supplied from the electrodes 106 to the determination unit 114, the determination unit 114 may put the puzzle pieces together to derive information regarding the DNA 102 sequence.

In the following, some recognitions of the present inventor regarding the conventional Sanger method will be explained referring to FIG. 2 to FIG. 5, and based on these considerations exemplary embodiments of the invention have been derived.

The dideoxy sequencing (also called chain termination or Sanger method) uses an enzymatic procedure to synthesize DNA chains of varying length, stopping DNA replication at one of the four bases and then determining the resulting fragment length. Each sequencing reaction tube of a conventional Sanger method (named ddT tube, ddC tube, ddG tube and ddA tube) may contain:
a collection of DNA templates (unknown sequence), a collection of primer sequences (one per template), and a DNA polymerase (one per primer and template) to initiate synthesis of a new strand of DNA at the point where the primer is hybridized to the template;
a sufficiently high concentration of the four nucleotides (A, T, C, and G) to extend the DNA primer strand complementary to the template;
a sufficiently low concentration of one of the four dideoxynucleotides, which terminates the growing chain wherever it is incorporated. For instance, tube ddA has ddA, tube ddC has ddC, tube ddG has ddG, and tube ddT as ddT.

FIG. 2 again shows an oligonucleotide primer 108 and an unknown DNA sequence (template) 102. The individual bases of the DNA sequence 102 are denoted with A (adenine), G (guanine), C (cytosine), T (thymine). As can be taken from FIG. 2, the olignucleotide primer 108 is complementary to a part of the DNA sequence 102. When adding DNA polymerase and A, T, G, C, these components may be inserted into four distinct tubes 202, 204, 206, 208. In tube 202, ddA is added, in tube 204, ddG is added, in tube 206, ddC is added and in tube 208, ddT is added. Thus, four solutions are prepared containing the elements mentioned above, and the DNA polymerase is enabled to actuate.

The DNA synthesis may then be done by an enzyme (DNA polymerase) that adds nucleotides to the 3′-end of the primer 108 DNA chains towards the 5′ end of the DNA 102.

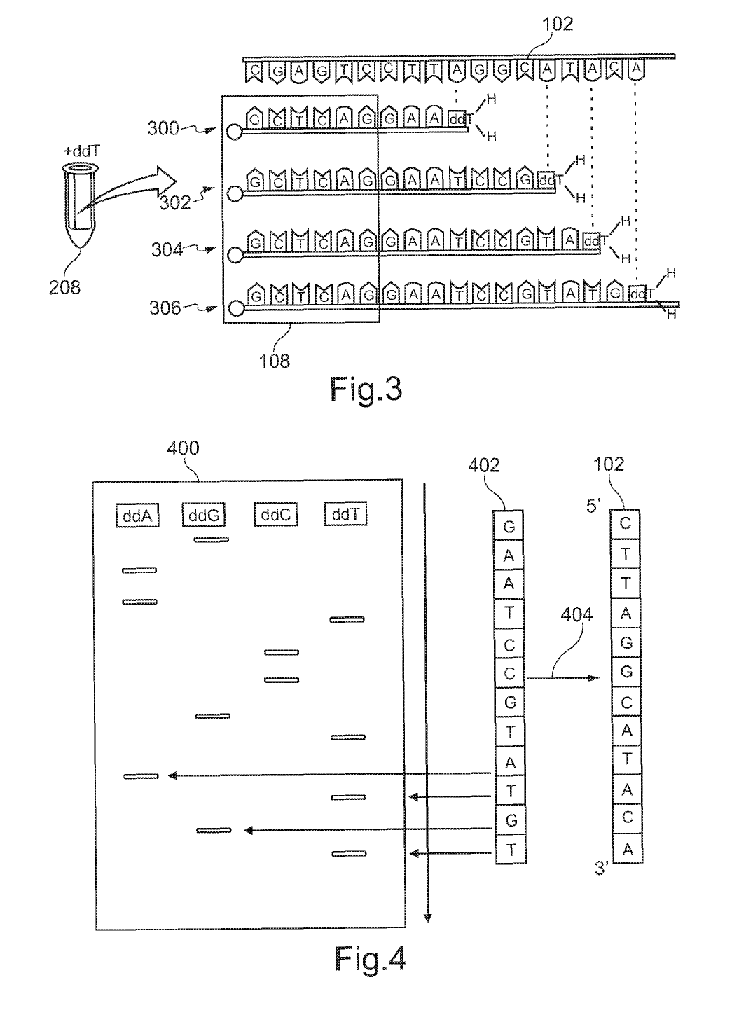

It is possible to stop the polymerase (that is DNA application) reaction when using dideoxynucleotides. Dideoxynucleotides are almost identical to the normal nucleotides. However, addition of a dideoxynucleotide to the 3′-OH end of the DNA chains stops the action of the DNA polymerase and terminates chain elongation. This is shown for the exemplary case of the tube 208 including the ddT in FIG. 3. First to fourth fragments 300, 302, 304, 306 are generated.

The polymerase starts adding nucleotides along the primer 108 that are complementary to the DNA template 102 until it incorporates a ddT. Then it stops. The result in the ddT tube 208 is a collection of fragments 300, 302, 304, 306 of different lengths of the DNA template ending always with a ddT. The result of the other three tubes 202, 204, 206 will be analogous except that all fragments end in ddA, ddG or ddC, respectively.

FIG. 4 shows how to derive the DNA sequence with a conventional Sanger method.

FIG. 4 shows the result 400 of a gel electrophoresis analysis of the fragments for ddA, ddG, ddC, and ddT. Furthermore, the sequence of the synthesized DNA 402 is shown which is complementary to the sequence of the template DNA 102. By an inverse conversion or complementary operation indicated schematically with reference number 404, the sequence of the template DNA 102 can be derived unambiguously from the sequence of the synthesized DNA 402.

All the solutions are run into an electrophoresis gel when the polymerase reaction stop. All the fragments are dragged by the electric field according to their length. Because the chain terminations (ddA, ddT, ddC, ddG) are known, it is possible to reconstruct the template sequence by reading the gel. The template sequence is complementary to the one that has been read in the gel.

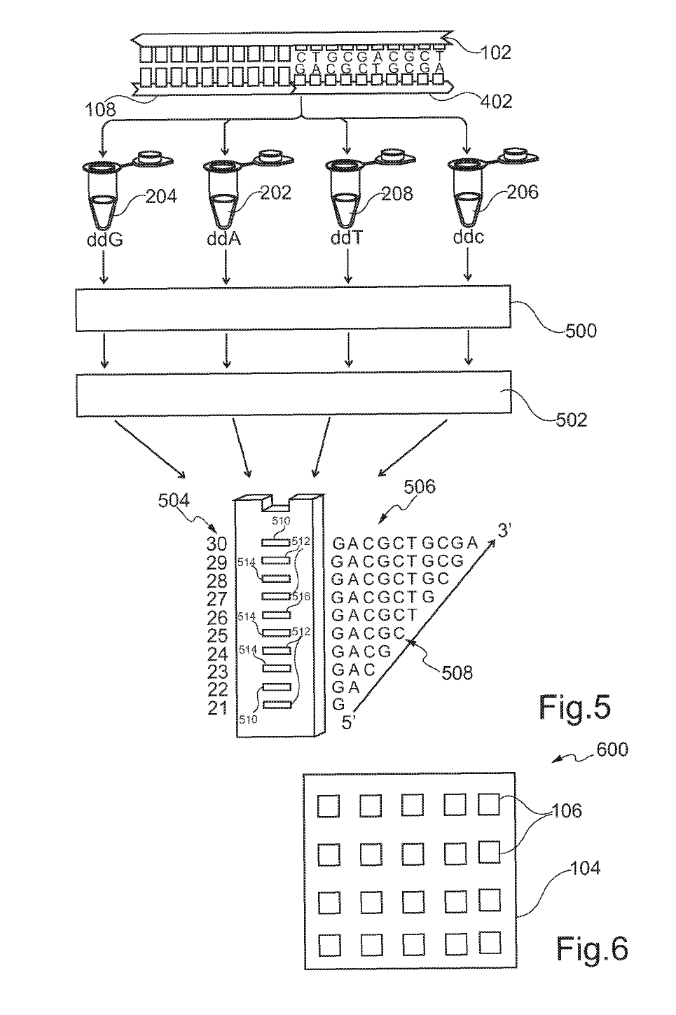

FIG. 5 again shows the result for the case of a primer of 20 bp.

With the embodiment of FIG. 5, the same as shown in FIG. 2 to FIG. 4 can be done in a single run when different fluorescence tags are used for every dd nucleotide. In a procedural step 500, synthesis continues until dideoxynucleotide (ddG, ddA, ddT, or ddC) is incorporated. In a procedural step 502, electrophoresis of the products is performed in a downward direction. The result is shown as the length of fragment 504 as well as the termination by dideoxy 506. As indicated by an arrow 508, the sequence is complementary to the DNA template strand 102. Different fluorescence labels 510, 512, 514, 516 are used for each of the nucleobases.

FIG. 6 shows a plan view of a biosensor device 600 according to an exemplary embodiment of the invention.

On a single substrate 104, a plurality of nanoelectrodes 106 is arranged in a matrix-like manner, that is to say in rows and columns. Without wishing to be bound to a specific theory, it is presently believed that each nanoelectrode 106 is sensitive enough to detect a single nucleotide incorporation to the primer 108 by capacitance changes. Each signal received from each nanoelectrode 106 is calibrated previously in a way that it can be discriminated when a single one, two or several nucleotides are added to the DNA primers 108.

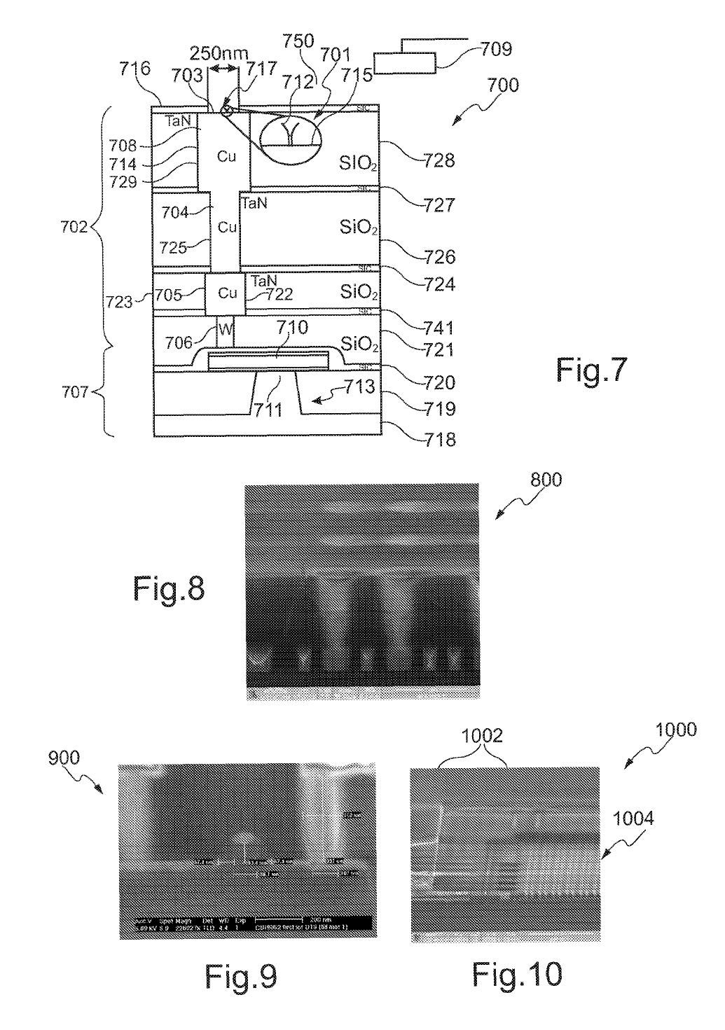

FIG. 7 shows a cross-sectional view of the biosensor device 700 according to an exemplary embodiment of the invention.

FIG. 7 is an example of a device 700 that can be used and implement the electronic Sanger method.

The biosensor chip 700 is adapted for detecting biological particles 12 and comprises a sensor active region 701 being sensitive for the biological particles 102 and being arranged on top of a Back End of the Line portion 702 of the biosensor chip 700. More particularly, the sensor active region 701 is arranged at an upper surface 703 of the BEOL region 702 of the biosensor chip 700.

A plurality of intermediate metallization structures 704 to 706 in the BEOL portion 702 are provided so that the sensor active region 701 is electrically coupled to a Front End of the Line (FEOL) portion 707 of the biosensor chip 700 via the plurality of intermediate metallization structures 704 to 706.

More particularly, a nanoelectrode 708 forming part of the sensor active region 701 is electrically coupled via the plurality of intermediate metallization structures 704 to 706 to a field effect transistor 713 integrated in the FEOL region 707.

A capacitor structure is partially formed in the Back End of the Line portion 702 and is arranged such that a capacitance value of the capacitor is influencable by a detection event at the sensor active region 701 (that is by a generation of fragments, not shown, to biological molecule-primer complexes 712 immobilized on the surface 703 of the sensor active region 701), since such a detection event may have an impact on the value of the permittivity in a sensor pocket 717. More particularly, the copper layer 708 forms a first electrode of such a capacitor, and a second electrode of this capacitor is formed by an electrolyte 750, connected by a counter electrode 709, which is, in the present embodiment, provided apart from the monolithically integrated layer sequence 700. Alternatively, it is possible to integrate an electrically conductive structure forming the second electrode of the capacitor in the layer stack.

More particularly, the actual capacitor in the biosensor 700 according to the exemplary embodiment of the invention is an electrolytic capacitor. The sensor 700 is immersed in an electrolyte 750 during the measurement. The electrolyte 750 can be the analyte itself or another conducting fluid that replaces the analyte after an experiment. The copper nano-electrode 708 is one capacitor plate, the conducting fluid 750 is the other capacitor “plate”. The two plates 708, 750 are separated by the SAM 715, which may contribute to the dielectric of the capacitor. When biological molecule-primer complexes 712 are attached to the SAM 715, the dielectric properties of the capacitor's dielectric will change, and consequently also the capacitance of the capacitor. The electrolyte 750 is connected with the counter electrode 709.

As schematically indicated in FIG. 7, the transistor structure 713 is formed in the Front End of the Line portion 707 and is electrically coupled to the sensor active region 701 via the plurality of metallization structures 704 to 706, 708. A gate region 710 of such a transistor 713 is shown, as well as a channel region 711. Source/drain regions are located in front of and behind the plane of the drawing, respectively, and therefore are not indicated explicitly in FIG. 7. They may be formed as doped regions electrically coupled to both sides of the channel region 711, as known by the skilled person.

As can be taken from FIG. 7, a single biological molecule-primer complex 712 is immobilized at a surface 703 of the sensor active region 701 and is adapted for interacting with biological particles.

The copper metallization structure 708 may have, at the surface 703, a dimension of 250 nm and therefore forms a nanoelectrode at which a detection event may take place. The nanoelectrode 708 is formed of copper material lined with a tantalum nitride layer 714. As can further be taken from FIG. 7, a SAM layer 715 (self assembled monolayer) is bridging the copper structure 708 and the biological molecule-primer complex 712.

The bare copper surface that remains after the final CMP step may oxidize rapidly in air or water. Therefore usually BTA (a corrosion inhibitor) is deposited during this CMP step (or during the subsequent cleaning step) to suppress this oxidation. In this way the wafers can be stored for some time (several days or perhaps even weeks) before the SAM 715 is deposited.

Just before the SAM deposition, the BTA has to be removed from the copper surface. Experimentally it is found that some wet-chemical SAM deposition recipes actually remove BTA themselves. In that case it is not strictly necessary to remove the BTA before the SAM deposition because it will happen automatically. After the SAM deposition it is not possible to deposit BTA anymore because the BTA would contaminate the SAM surface. Instead, a proper SAM 715 should act as a corrosion inhibitor by itself. Or the sensor chips have to be stored in a non-oxidizing atmosphere after the SAM deposition.

Beyond this, the biosensor chip 700 comprises an electrically insulating layer 716 forming part of a surface of the biosensor chip 700 and having a recess 717, wherein an exposed surface 703 of the sensor active region 701 is provided as a sensing pocket volume in the recess 717.

The biosensor chip 700 is manufactured in CMOS technology, starting from a silicon substrate 718, the surface of which is shown in FIG. 7, and which may have a P well or an N well.

Bond pads for electrically contacting the biosensor chip 700 may be provided but is not shown in FIG. 7.

More particularly, an electrically insulating shallow trench insulation structure 719 is provided on/in the semiconductor substrate 718. The gate 710 comprises polysilicon material and a CoSi silicide structure. Furthermore, a silicon carbide layer 720 is provided on the shallow trench insulation layer 719 and on the gate stack 710. A silicon oxide layer 721 has a contact hole in which the tungsten contact 706 is formed. On top of this structure, a further silicon carbide layer 741 is provided. On top of the silicon carbide layer 741, a tantalum nitride liner 722 is foreseen to line a trench, filled with copper material to form the copper metal structure 705. This is embedded in a further silicon oxide layer 723. On top of this structure, a further silicon carbide layer 724 is formed, followed by forming a tantalum nitride liner 725 in a via hole formed in a further silicon oxide layer 726. The lined via hole is filled with copper material, thereby forming the copper via 704. Next, a silicon carbide layer 727 may be deposited, followed by the position of a further silicon oxide layer 728, in which a further trench may be etched which may be lined with an additional tantalum nitride structure 729. This lined trench may be filled with copper material, thereby forming the copper metal layer 708.

A CMP (chemical mechanical polishing) procedure may be carried out to generate the essentially planar surface in the biosensor chip 700.

FIG. 8 shows an image 800 illustrating an example of a nanoelectrode.

FIG. 9 gives an example of a transistor 900.

FIG. 10 shows an image 1000 illustrating a top view of the device 700 showing the plurality of nanoelectrodes. A scratch protection access area 1002 is shown as well as the array 1004 of electrodes.

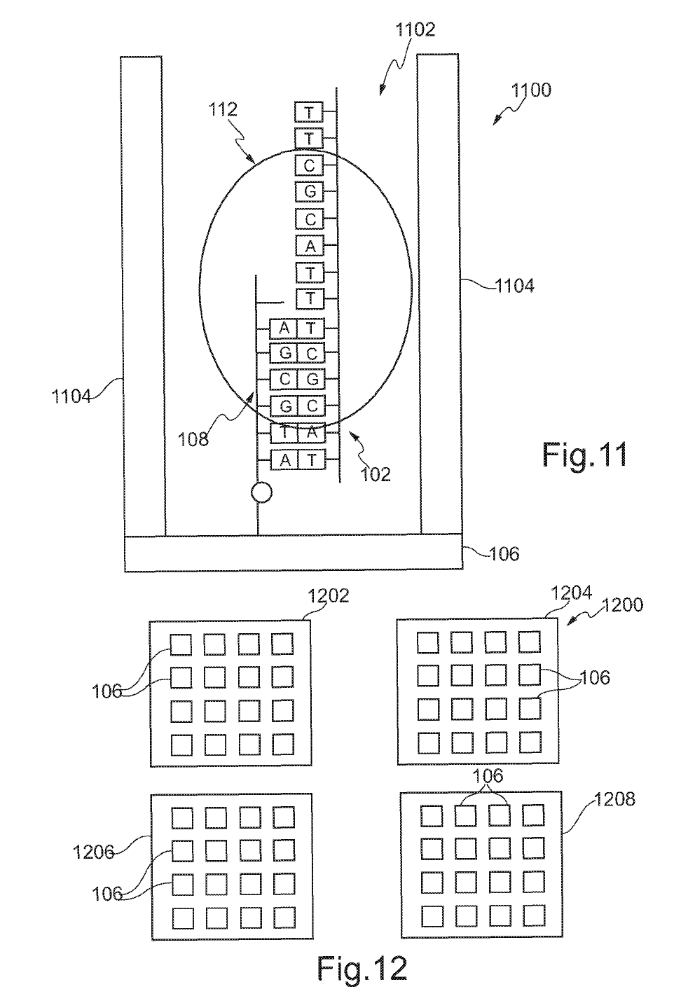

FIG. 11 shows an enlarged view 1100 of a sensor active region 106. Within a sensing pocket 1102 which may be a trench or the like and which may be delimited by electrically insulating walls 1104, the primer 108 may be immobilized at the electrode 106. The unknown DNA chain (template) 102 is shown as well. Furthermore, a DNA polymerase 112 is shown. It is possible to incorporate in each nanoelectrode 106 the primer 108 and the unknown DNA chain 102. A, T, G, C and ddA, ddG, ddT and ddC may be floating in solution (not shown in FIG. 11).

FIG. 12 shows a biosensor device 700 having a first substrate 1202, a separate second substrate 1204, a separate third substrate 1206 and a separate fourth substrate 1208. A plurality of matrix-like arranged nanoelectrodes 106 are shown on a surface of each of the substrates 1202, 1204, 1206, 1208.

On the first chip 1202, A, T, G, C and ddA, as well as polymerase, primer and an unknown DNA sequence are supplied. To the second chip 1206, A, T, G, C, ddT, polymerase, primer, and an unknown DNA sequence is added. To the third chip 1204, A, T, G, C, ddG, polymerase, primer, and an unknown DNA sequence is added. To the fourth chip 1208, A, T, G, C, ddC, polymerase, primer and an unknown DNA sequence are added.

As can be taken from FIG. 12, each nanoelectrode 106 is exposed to the indicated solution. The polymerase acts like in the Sanger method.

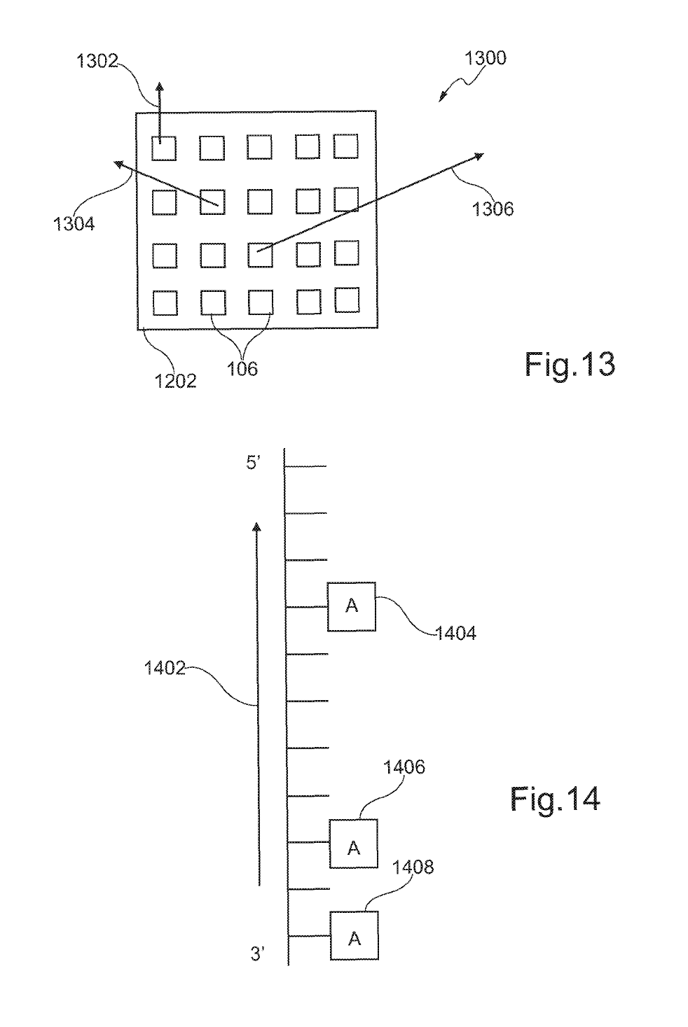

FIG. 13 shows an image 1300 again showing the first chip 1202.

FIG. 13 is an example of the kind of information obtained from reading the first chip 1202. As indicated by reference numeral 1302, no nucleotide is incorporated and stopped on ddA on electrode (1,1). Two nucleotides are incorporated and stopped on ddA on electrode (2, 2), as indicated by reference numeral 1304. 7 nucleotides are incorporated and stopped on ddA on electrode (3, 3), as indicated by reference numeral 1306. Thus, each electrode 106 receives a distinctive capacitive signal proportional to the number of nucleotides incorporated. The sequence shown in FIG. 13 is only exemplary. Any other combination is possible, for example electrode (1,1) 0 nucleotides, electrode (1, 2) 2 nucleotides, electrode (1, 3) 7 nucleotides.

FIG. 14 schematically illustrates an example of the kind of information obtained reading the first chip 1202. In this example, ddA counts as a nucleotide. As indicated by an arrow 1402, the polymerase incorporates nucleotides to the primer in this direction always. As indicated by reference numeral 1404, electrode (3,3) has 7 nucleotides incorporated and stops on ddA. As indicated by reference numeral 1406, electrode (2,2) has two nucleotides incorporated and stops on ddA. As indicated by reference numeral 1408, electrode (1,1) has zero nucleotides incorporated and stops on ddA.

Therefore, all positions of “A” in the sequence may be derived from the first chip 1202.

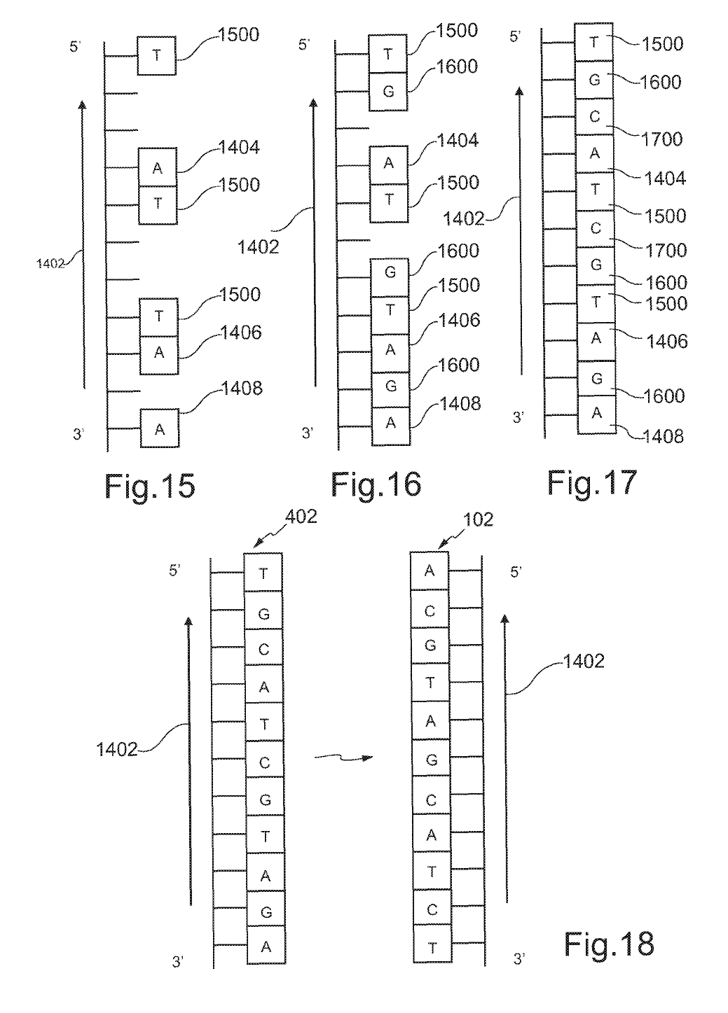

FIG. 15 illustrates how information is obtained after reading the second chip 1206. From the fragments, the positions of the “T” may be derived, as indicated by reference numeral 1500.

FIG. 16 shows which information can be derived after reading the third chip 1204. As indicated by reference numeral 1600, the positions of the “G” can be derived.

FIG. 17 shows which information can be derived after reading the fourth chip 1208. As indicated by reference numeral 1700, information regarding the “C” can be derived.

As can be taken from FIG. 18, the unknown DNA chain 102 is reconstructed after having read out the four chips 1202, 1204, 1206, 1208. The DNA chain built by the primer is denoted with reference numeral 402, whereas the previously unknown DNA chain 102 is complementary to the primer chain 402.

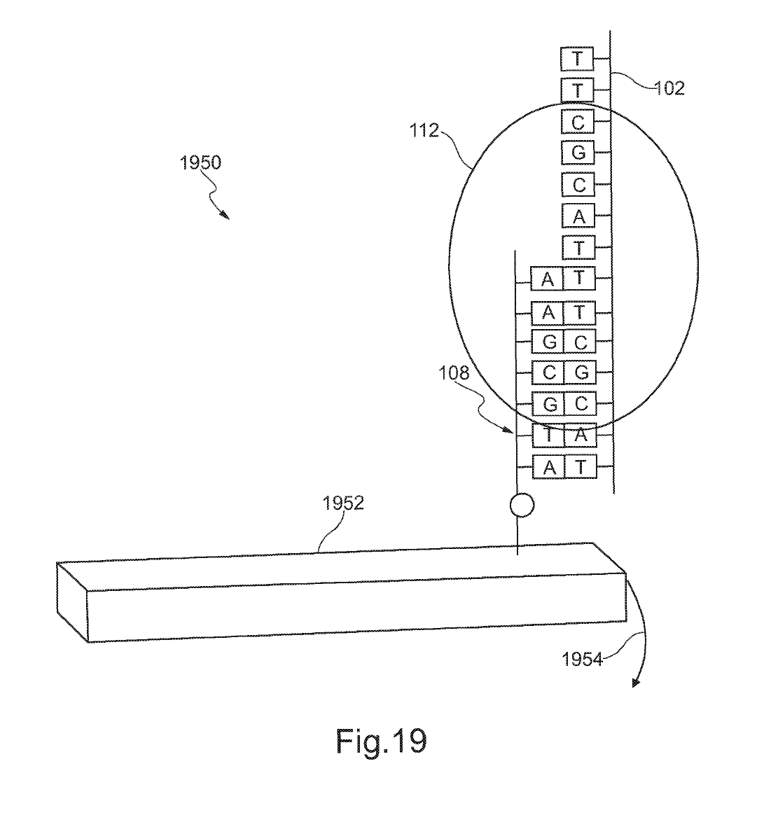

FIG. 19 shows a biosensor device 1950 according to another exemplary embodiment of the invention.

As compared to FIG. 11, a nanocantilever 1952 is shown as a sensing element instead of a nanoelectrode. The nanocantilever 1952 is bendable (see arrow 1954) under the mechanical force of the attached molecules 108, 102. Nucleotides and dideoxynucleotides are floating in a solution, as in FIG. 11. Each nanocantilever 1952 may have attached the polymerase 112, the primer 108 and the template 102.

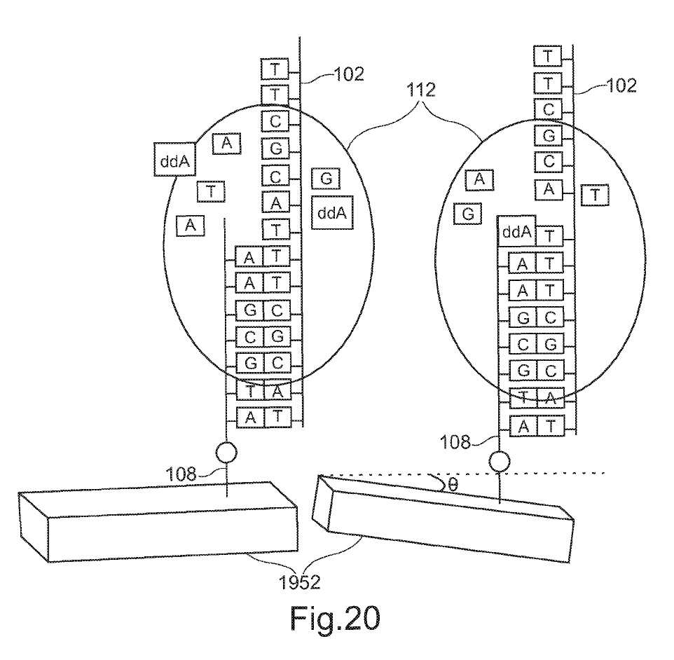

As indicated schematically in FIG. 20, when a ddA arrives, the polymerase reaction stops. The cantilever 1952 experiments a deflection that can be proportional to the mass that has been added. Therefore, with the cantilever 1952 being mounted in a bendable manner, it is possible to derive, from the extent of the bending, information indicative of the length of the added fragment.

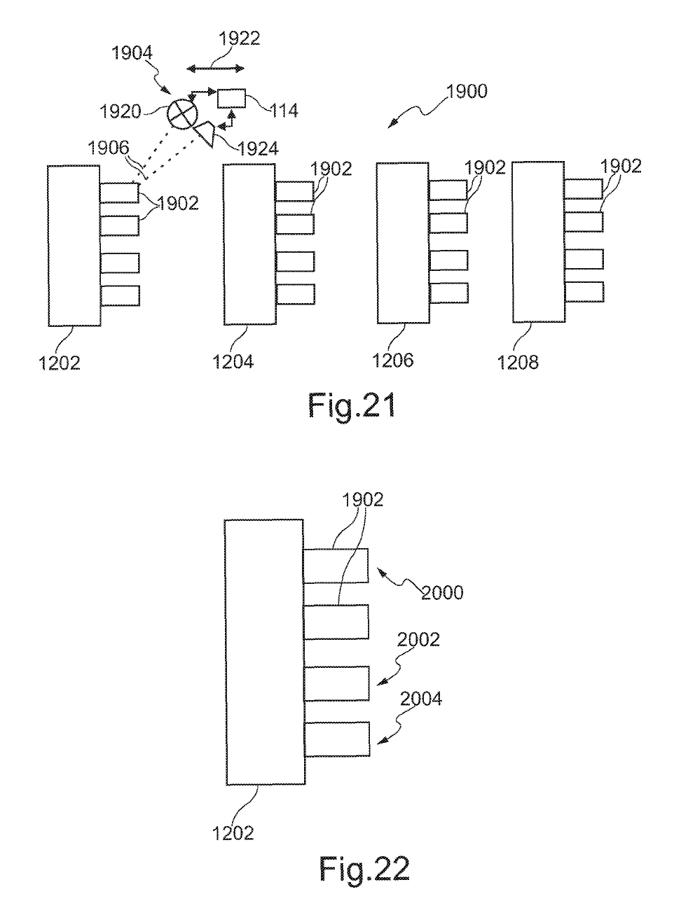

FIG. 21 illustrates an electronic Sanger biosensor device 1900 according to an exemplary embodiment of the invention.

In FIG. 21, four nanocantilever arrays are shown each having a substrate 1202, 1204, 1206, 1208 and attached cantilevers 1902. The nanocantilever array connected to the substrate 1202 is provided with A, T, G, C, ddA, polymerase, primer, and unknown DNA sequence. The nanocantilever array assigned to the substrate 1204 is provided with A, T, G, C, ddT, polymerase, primer, and an unknown DNA sequence. The nanocantilever array connected with the substrate 1206 is provided with A, T, G, C, ddG, polymerase, primer, and an unknown DNA sequence. The nanocantilever array assigned to the substrate 1208 is supplied with A, T, G, C, ddC, polymerase, primer, and an unknown DNA sequence. As can be taken from FIG. 21, the determination unit 114 here also acts as a control unit. The control unit 114 controls an exciting laser 1920, which directs a light beam 1906 to a specific one of the cantilevers 1902. As indicated with an arrow 1922, the laser 1920 can scan the entire arrangement 1900. A photodiode or CCD detector 1924 detects the reflected light to derive reflection properties and therefore calculates a deflection of the cantilevers 1902.

FIG. 22 schematically illustrates, for the nanocantilever array assigned to the substrate 1202, how information can be derived. As indicated with reference numeral 2000, the nanocantilever (1,1) has 0 nucleotides incorporated and stops on ddA. The nanocantilever 2002 (2,2) has 2 nucleotides incorporated and stops on ddA. The nanocantilever (3,3) has seven (7) nucleotides incorporated and stops on ddA, as shown by reference numeral 2004.

A laser may be directed towards each one of the cantilevers 1902 in the array shown in FIG. 21, and the deflection value is read, which is representative to the number of nucleotides incorporated to the primers. Thus, in the same manner as shown in FIG. 14 to FIG. 18, the DNA sequence can be derived from the cantilever bending. A proportional relationship may be given between the deflection value and the number of nucleotides incorporated.

Finally, it should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The words “comprising” and “comprises”, and the like, do not exclude the presence of elements or steps other than those listed in any claim or the specification as a whole. The singular reference of an element does not exclude the plural reference of such elements and vice-versa. In a device claim enumerating several means, several of these means may be embodied by one and the same item of software or hardware. The mere fact that certain measures are recited in mutually different dependent claims does not indicate that a combination of these measures cannot be used to advantage.

1. A biosensor device for sequencing biological particles, the biosensor device comprising
at least one substrate;
a plurality of sensor active regions provided on each of the at least one substrate and each of the sensor active regions comprising a primer having a sequence being complementary to a part of a sequence of the biological particles for enabling generation of fragments having a sequence being inverse to a part of the sequence of the biological particles at the primer;
a determination unit adapted for individually determining a size of the fragments replicated at the primer of each of the plurality of sensor active regions, the fragment generation being terminated in the presence of replication terminating sequence units.

2. The biosensor device of claim 1, wherein the determination unit is adapted for determining the sequence of the biological particles based on the individually determined size of the fragments considering information regarding an assigned type of replication terminating sequence units.

3. The biosensor device of claim 1, wherein the at least one substrate consists of exactly one substrate having delimited compartments, each of the delimited compartments comprising a plurality of the sensor active regions and being assigned to a type of the replication terminating sequence units.

4. The biosensor device of claim 1, wherein the at least one substrate comprises a plurality of separate substrates, each of the separate substrates comprising a plurality of the sensor active regions and being assigned to a type of the replication terminating sequence units.

5. The biosensor device of claim 1, wherein the plurality of sensor active regions comprise electrodes.

6. The biosensor device of claim 5, wherein an exposed surface of the electrodes has a dimension of less than about 300 nm.

7. The biosensor device of claim 5, wherein the electrodes comprise copper material.

8. The biosensor device of claim 5, wherein the electrodes form a capacitor structure arranged such that a capacitance value of the capacitor is influenced by a detection event in the corresponding sensor active region.

9. The biosensor device of claim 5, wherein the determination unit is adapted for evaluating electric signals received at the electrodes, the electrical signals being indicative of the assigned size of fragments.

10. The biosensor device of claim 1, wherein the plurality of sensor active regions comprise cantilever beams, being bendable in a characteristic manner based on the size of fragments.

11. The biosensor device of claim 10, wherein the determination unit is adapted for sampling a bending of the cantilever beams using an electromagnetic radiation beam, deflection of the electromagnetic radiation beam at the bendable cantilever beams being indicative of the assigned size of fragments.

12. The biosensor device of claim 1, wherein an exposed surface of the sensor active region has a dimension of at most 1.6 times, of a minimum lithographic feature size of a CMOS process applied for manufacturing the biosensor device.

13. The biosensor device of claim 1, comprising a switch transistor structure electrically coupled to the sensor active region.

14. The biosensor device according to claim 1, manufactured in CMOS technology or in MEMS technology.

15. The biosensor device according to claim 1, being monolithically integrated in a semiconductor substrate, comprising one of the group consisting of a group IV semiconductor, and a group III-group V semiconductor.

16. The biosensor device according to claim 1, wherein the primer is adapted for enabling generation of fragments having a sequence being inverse to a part of the sequence of the biological particles at the primer in the presence of sequence units of the sequence of the biological particles and in the presence of a replication enzyme.

17. A method of sequencing biological particles, the method comprising
providing a plurality of sensor active regions on each of at least one substrate, each of the plurality of sensor active regions comprising a primer having a sequence being complementary to a part of a sequence of the biological particles and enabling generation of fragments having a sequence being inverse to a part of the sequence of the biological particles at the primer;
individually determining a size of fragments generated at the primer of each of the plurality of sensor active regions, the fragment replication being terminated in the presence of replication terminating sequence units.

18. The method according to claim 17, comprising determining the sequence of the biological particles based on the individually determined size of the fragments considering information regarding an assigned type of replication terminating sequence units.

19. The biosensor device of claim 3, wherein the at least one substrate consists of exactly one substrate having four delimited compartments.

20. The biosensor device of claim 4, wherein the plurality of separate substrates is four.
